(19) United States
(12) Patent Application Publication
(10) Pub. No.: US 2025/0005451 A1
(43) Pub. Date: Jan. 2, 2025

(54) COMPOSITION SEARCH METHOD

(71) Applicant: Resonac Corporation, Tokyo (JP)

(72) Inventors: Haein LEE, Tokyo (JP); Takuya MINAMI, Tokyo (JP); Yoshishige OKUNO, Tokyo (JP)

(21) Appl. No.: 18/694,641

(22) PCT Filed: Sep. 28, 2022
(86) PCT No.: PCT/JP2022/036163

§ 371 Date: Mar. 22, 2024

(30) Foreign Application Priority Data
Oct. 4, 2021 (JP) 2021-163338

(51) Int. Cl. G06N 20/00

(52) U.S. Cl. CPC G06N 20/00

(57) ABSTRACT

A composition search method for a material includes constructing a prediction model by learning training data in which information related to a composition of a material is set as an explanatory variable and a value of a physical property of the material is set as an objective variable; calculating a predicted value of the physical property by inputting, into the prediction model, prediction data for newly searching for a composition; calculating an influence degree of each explanatory variable on prediction by using the training data and the prediction model; calculating a weighted distance of the prediction data with respect to the training data by using the influence degree; and displaying a relationship between the predicted value and the weighted distance, and outputting corresponding prediction data as a search candidate.

### TECHNICAL FIELD

The present invention relates to a composition search method.

### BACKGROUND

In material design, it is necessary to determine parameters (a composition or a composition ratio, and a constraint condition such as cost and a manufacturing condition) for obtaining a value of a physical property of a target material.

Conventionally, an experimenter often determines parameters empirically or by trial and error. However, in a case of a complicated material design with a large number of parameters, it takes a long time and is extremely difficult to obtain a target physical property.

In order to improve such a conventional material design, a technique of obtaining an optimum parameter by performing machine learning using accumulated data in which the above-described parameters are associated with known physical properties is proposed in recent years.

As an example, Patent Document 1 below proposes an optimization method of generating a Bayesian model for searching for a combination of values of multiple parameters that gives an optimum value as a value of a physical property related to a target substance, and performing a search for the combination using the Bayesian model in a search space.

Additionally, Non-Patent Document 1 below proposes a technique that is one of sequential search methods using a prediction model, that determines a next candidate point by using a distance between a prediction value and a training data value, and that optimizes a hyperparameter of the model. According to this method, the prediction method is not limited in the parameter search.

As another example, Patent Document 2 below proposes searching for a design condition so as to reduce variations in multiple predicted values that are obtained based on multiple different training datasets, when searching for a parameter in which a desired physical property can be obtained, by using a prediction model that predicts a value of a physical property from a design parameter of a metallic material, searching for a parameter including a new region different from past actual data so as to increase a difference between the parameter and a parameter in the past actual data.

### RELATED ART DOCUMENT

### Patent Document

Patent Document 1: Japanese Laid-open

Patent Application Publication No. 2020-187642

Patent Document 2: International Publication No. WO 2010-152993

### Non-Patent Document

Non-Patent Document 1: DOI: arxiv-2101.02289

### SUMMARY OF THE INVENTION

### Problem to be Solved by the Invention

However, the invention disclosed in Patent Document 1 uses a Bayesian model, and is limited to an optimization method of Gaussian process regression. Therefore, there is a problem that another prediction method (for example, gradient boosting, a neural network, or the like) expected to have high prediction performance cannot be flexibly used, and the prediction method is limited.

In the technique described in Non-Patent Document 1, the prediction method is not limited in the parameter search. In the case of the technique described in Non-Patent Document 1, prediction accuracy verified with past parameters is weighted on a term of a distance from training data so that a parameter away from the past parameters can be searched for in consideration of the accuracy of the prediction model. However, in the case of the technique described in Non-Patent Document 1, the weighting is uniformly performed on all parameters, and a search is uniformly performed including a parameter having a small relationship with an objective variable. Therefore, there is a problem that it takes time to reach the optimum parameter.

Additionally, the invention disclosed in Patent Document 2 is configured to apply a weight to each parameter so that a difference from a parameter in past actual data increases, but the weight is determined by a user, which is arbitrary, and therefore, there is a problem that the search is not necessarily performed appropriately.

It is an object of the present invention to provide a composition search method of searching for a composition for obtaining a target value of a physical property more efficiently.

### Means for Solving the Problem

The present invention has the following configurations.

[1] A composition search method for a material including:
a step of constructing a prediction model by learning training data in which information related to a composition of a material is set as an explanatory variable and a value of a physical property of the material is set as an objective variable;
a step of calculating a predicted value of the physical property by inputting, into the prediction model, prediction data for newly searching for a composition;
a step of calculating an influence degree of each explanatory variable on prediction by using the training data and the prediction model;
a step of calculating a weighted distance of the prediction data with respect to the training data by using the influence degree; and
a step of displaying a relationship between the predicted value and the weighted distance, and outputting corresponding prediction data as a search candidate.

[2] The composition search method as described in [1], wherein in the step of calculating the weighted distance, the weighted distance is scaled to a value between zero and one, inclusive.

[3] The composition search method as described in [1] or [2],
wherein the prediction data are a combination of information related to the composition exhaustively generated according to a constraint condition of a step size or a composition ratio that is set in advance, and
wherein by repeating the step of calculating the predicted value of the physical property to the step of calculating the weighted distance, in the step of displaying the relationship between the predicted value and the weighted distance, a plurality of said relationships between the calculated predicted values and the weighted distances are displayed.

[4] The composition search method as described in [3], further including a step of grouping the predicted values by the weighted distances, and
wherein in the step of displaying the relationship between the predicted value and the weighted distance, the prediction data are divided into groups and output.

[5] The composition search method as described in [4], wherein in the step of displaying the relationship between the predicted value and the weighted distance, corresponding prediction data are output as search candidates in an order in which the predicted value is higher for each of the groups.

[6] The composition search method as described in [4] or [5], wherein in the step of grouping, the grouping is performed by equally dividing the weighted distances by a predetermined value between zero and one.

[7] The composition search method as described in [4] or [5], wherein in the step of grouping, the grouping is performed by dividing the weighted distance between zero and one such that a number of the predicted values in a group after the division is identical.

[8] The composition search method as described in any one of [3] to [6], wherein in the step of displaying the relationship between the predicted value and the weighted distance, a number of the prediction data to be output as the search candidate is set by a user.

[9] The composition search method as described in [4], further including:
a step of calculating an acquisition function Acq (Xᵢ) with respect to the predicted value and the weighted distance calculated from the prediction data by using the following Equation (1); and
a step of outputting corresponding prediction data as the search candidates in an order in which the calculated acquisition function is higher.

$$\begin{matrix}[ Eq . 1 ] & \\ Acq ( {X}_{i} ) = ( 1 - {s}_{g} ) * f ( {X}_{i} ) + {s}_{g} * {D}_{i} & ( 1 )\end{matrix}$$

$$( 0 \leq {s}_{g} \leq 1 )$$

Here, Xᵢ is the i-th prediction data, f (Xᵢ) is a predicted value of Xᵢ scaled to a value between zero and one, inclusive, S_{g} is a weighting factor in the g-th group, and Dᵢ is the weighted distance of Xᵢ.

[10] The composition search method as described in [3], further including:
a step of performing an experiment based on the information related to the composition of the prediction data output as the search candidate in the step of outputting, to obtain a value of the physical property; and
a step of adding the information related to the composition corresponding to the obtained value of the physical property to the training data,
wherein processing of constructing the prediction model by using the training data to which data is added in the step of constructing the prediction model to processing of obtaining the value of the physical property in the step of obtaining the value of the physical property are repeated until the obtained value of the physical property reaches a predetermined target value.

### Effect of the Invention

According to the present disclosure, a composition for obtaining a target value of a physical property can be searched for more efficiently.

### BRIEF DESCRIPTION OF THE DRAWINGS

FIG. 1 is a first diagram illustrating an example of a system configuration of a composition search system.

FIG. 2 is a diagram illustrating an example of a hardware configuration of a learning device and a predicting device.

FIG. 3 is a diagram illustrating an example of training data and prediction data.

FIG. 4 is a first diagram illustrating an example of a graph indicating a relationship between a predicted value and a weighted distance.

FIG. 5 is a first flowchart illustrating a flow of a composition search process.

FIG. 6 is a second diagram illustrating an example of the system configuration of the composition search system.

FIG. 7 is a second diagram illustrating an example of the graph indicating the relationship between the predicted value and the weighted distance.

FIG. 8 is a third diagram illustrating an example of the graph indicating the relationship between the predicted value and the weighted distance.

FIG. 9 is a second flowchart illustrating a flow of the composition search process.

FIG. 10 is a third diagram illustrating an example of the system configuration of the composition search system.

FIG. 11 is a third flowchart illustrating a flow of the composition search process.

FIG. 12 is a graph indicating the number of times for ending of search in an example and comparative examples.

### DESCRIPTION OF THE EMBODIMENTS

In the following, each embodiment will be described with reference to the accompanying drawings. In order to facilitate understanding of the description, the same reference symbols are given to the same components in the drawings as far as possible, and duplicated description will be omitted.

### First Embodiment

A composition search method according to a first embodiment includes: a step of constructing a prediction model by learning training data in which information related to a composition of a material is set as an explanatory variable and a value of a physical property of the material is set as an objective variable; a step of calculating a predicted value of the physical property by inputting, into the prediction model, prediction data for newly searching for a composition; a step of calculating an influence degree of each explanatory variable on prediction by using the training data and the prediction model; a step of calculating a weighted distance of the prediction data with respect to the training data by using the influence degree; and a step of displaying a relationship between the predicted value and the weighted distance and outputting corresponding prediction data as a search candidate.

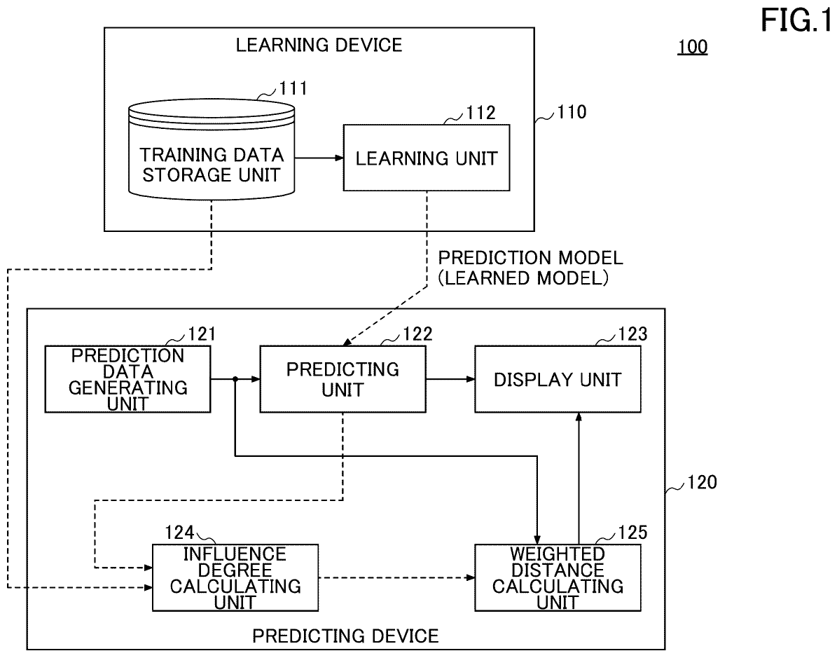

Here, in the present specification, the composition may be elements constituting an alloy material, or may be various raw materials constituting an organic material or a composite material. Additionally, in the present specification, a type, a preparation ratio, a feature, and the like of the raw material, which are information related to the composition, are also referred to as parameters of the raw material. Hereinafter, the details of the composition search method according to the first embodiment will be described using FIG. 1 to FIG. 5.

### System Configuration of Composition Search System

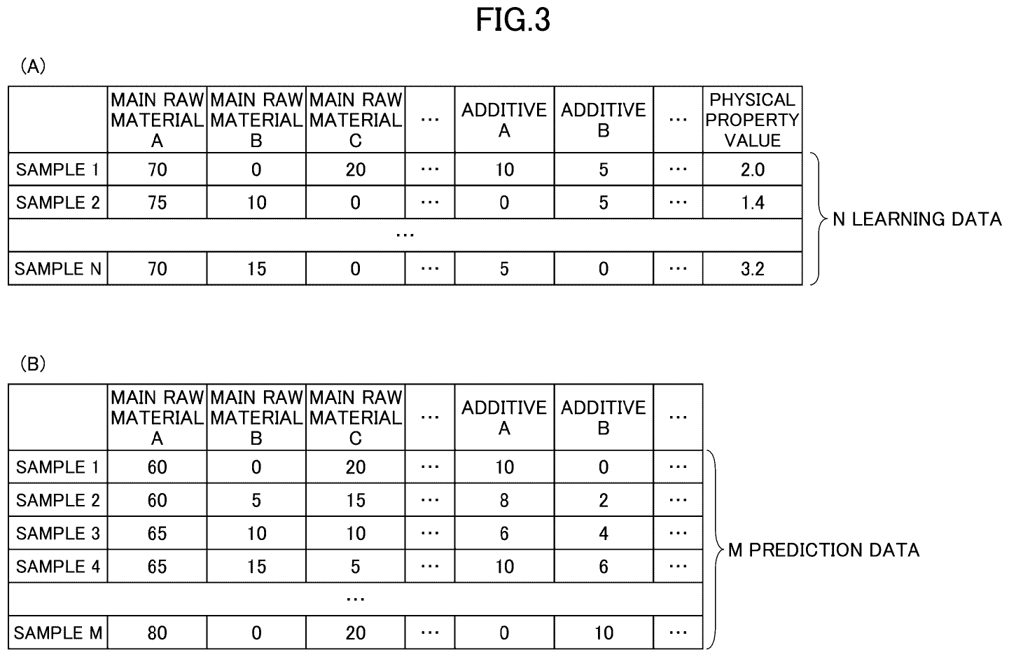

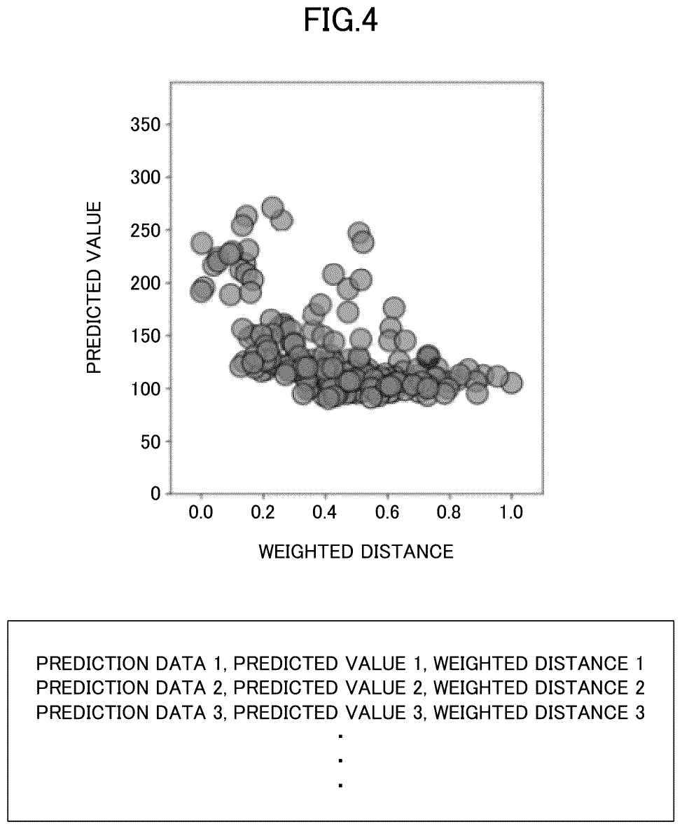

First, a system configuration of a composition search system for realizing the composition search method according to the first embodiment will be described using FIG. 1 with reference to FIG. 3 and FIG. 4. FIG. 1 is a first diagram illustrating an example of the system configuration of the composition search system. FIG. 3 is a diagram illustrating an example of the training data and the prediction data. FIG. 4 is a first diagram illustrating an example of a graph indicating the relationship between the predicted value and the weighted distance.

As illustrated in FIG. 1, a composition search system 100 includes a learning device 110 and a predicting device 120.

A learning program is installed in the learning device 110, and the learning device 110 functions as a learning unit 112 by executing the program.

The learning unit 112 constructs a prediction model (a learned model) by using the training data stored in a training data storage unit 111. In the present embodiment, the training data used when the learning unit 112 constructs the prediction model includes a set of the parameters of the raw material (the type, the preparation ratio, the feature) and a measured value of the physical property for multiple experimental samples (see FIG. 3 (A)).

Additionally, in the present embodiment, the model trained by the learning unit 112 includes any method such as random forest, Gaussian process regression, a neural network, and an ensemble learning model combining multiple methods.

Here, the prediction model (the learned model) constructed by the learning unit 112 is set in a predicting unit 122 of the predicting device 120.

A predicting program is installed in the predicting device 120, and the predicting device 120 functions as a prediction data generating unit 121, the predicting unit 122, a display unit 123, an influence degree calculating unit 124, and a weighted distance calculating unit 125 by executing the program.

The prediction data generating unit 121 generates the prediction data. The prediction data includes data of combinations of compositions exhaustively generated according to a constraint condition defining upper and lower limits and a step size of a composition ratio, raw materials that cannot be used at the same time, and the like, or features related to the compositions (see FIG. 3 (B)). Here, the prediction data generating unit 121 inputs the generated prediction data into the predicting unit 122 and notifies the weighted distance calculating unit 125 of the prediction data.

The predicting unit 122 calculates a predicted value from the prediction data by using the prediction model. Additionally, the predicting unit 122 notifies the display unit 123 of the calculated predicted value.

The influence degree calculating unit 124 calculates the influence degree of each explanatory variable on the prediction using the training data stored in the training data storage unit 111 and the prediction model. Specifically, the influence degree calculating unit 124 calculates the influence degree by using various algorithms stored in various Python libraries.

For example, when the prediction model is a linear model, the influence degree calculating unit 124 calculates the influence degree by using a coefficient of each variable. Additionally, when the prediction model is a model based on a decision tree, the influence degree calculating unit 124 calculates the influence degree, such as permutation importance or Gini importance. Alternatively, the influence degree calculating unit 124 may calculate the influence degree by using an algorithm of SAGE or SHAP of a Python library, which can calculate the influence degree in a selected method.

The weighted distance calculating unit 125 calculates the weighted distance of the prediction data with respect to the training data by using the influence degree calculated by the influence degree calculating unit 124. Specifically, the weighted distance calculating unit 125 calculates the weighted distance by using the following Equations (2) and (3).

$$\begin{matrix}[ Eq . 2 ] & \\ {d}_{n} = \sqrt{{\sum }_{t = 1}^{k} {w}_{t} \times {( {X}_{{n}_{t}} - {x}_{{n}_{t}} )}^{2}} & ( 2 )\end{matrix}$$

$$\begin{matrix}[ Eq . 3 ] & \\ {D}_{i} = \frac{1}{N} {\sum }_{n = 1}^{N} {d}_{n} & ( 3 )\end{matrix}$$

Here, dₙ is a weighted average distance between the n-th prediction data and the training data, N is the total number of the experiments in which measurements are performed, k is the total number of the explanatory variables (the parameters of the raw material), Xₙₜ is the t-th explanatory variable in the n-th training data, xₙₜ is the t-th explanatory variable in the n-th prediction data, and wₜ is the influence degree. The weighted distance Dᵢ is a value obtained by scaling the calculated dₙ to a value between zero and one, inclusive.

The display unit 123 displays multiple relationships between the prediction values calculated by the predicting unit 122 and the weighted distances calculated by the weighted distance calculating unit 125. For example, the display unit 123 displays multiple relationships between the predicted values and the weighted distances by using a two dimensional graph in which the horizontal axis represents the weighted distance and the vertical axis represents the predicted value (see FIG. 4). Additionally, the display unit 123 outputs the corresponding prediction data as the search candidate.

### Hardware Configuration of Learning Device and Predicting Device

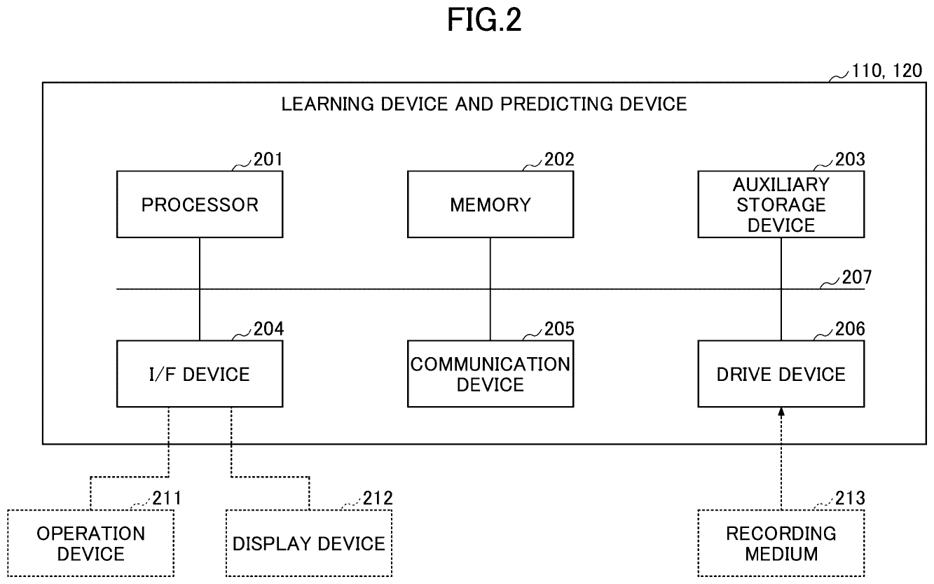

Next, a hardware configuration of the learning device 110 and the predicting device 120 included in the composition search system 100 will be described. Here, in the present embodiment, the hardware configuration of the learning device 110 and the hardware configuration of the predicting device 120 are substantially the same, and therefore, here, the configurations will be described together with reference to FIG. 2. FIG. 2 is a diagram illustrating an example of the hardware configuration of the learning device and the predicting device.

As illustrated in FIG. 2, the learning device 110 and the predicting device 120 include a processor 201, a memory 202, an auxiliary storage device 203, an interface (I/F) device 204, a communication device 205, and a drive device 206. Here, hardware components of each of the learning device 110 and the predicting device 120 are connected to each other via a bus 207.

The processor 201 includes various arithmetic devices, such as a central processing unit (CPU), a graphics processing unit (GPU), and the like. The processor 201 reads various programs (for example, a learning program, a predicting program, and the like) into the memory 202 and executes the programs.

The memory 202 includes a main storage device, such as a read only memory (ROM) or a random access memory (RAM). The processor 201 and the memory 202 form what is called a computer, and by the processor 201 executing various programs read into the memory 202, the computer realizes various functions.

The auxiliary storage device 203 stores various programs and various data used when the various programs are executed by the processor 201. For example, the training data storage unit 111 is realized in the auxiliary storage device 203.

The I/F device 204 is a connection device that connects to an operation device 211 and a display device 212, which are examples of user interface devices. The communication device 205 is a communication device for communicating with an external device (not illustrated) via a network.

The drive device 206 is a device in which a recording medium 213 is set. The recording medium 213 herein includes a medium for optically, electrically, or magnetically recording information, such as a CD-ROM, a flexible disk, or a magneto-optical disk. Additionally, the recording medium 213 may include a semiconductor memory or the like that electrically records information, such as a ROM or a flash memory.

Here, the various programs to be installed in the auxiliary storage device 203 are installed by, for example, the distributed recording medium 213 being set in the drive device 206 and the various programs recorded in the recording medium 213 being read by the drive device 206. Alternatively, the various programs to be installed in the auxiliary storage device 203 may be installed by being downloaded from the network via the communication device 205.

### Flow of Composition Search Process in Composition Search System

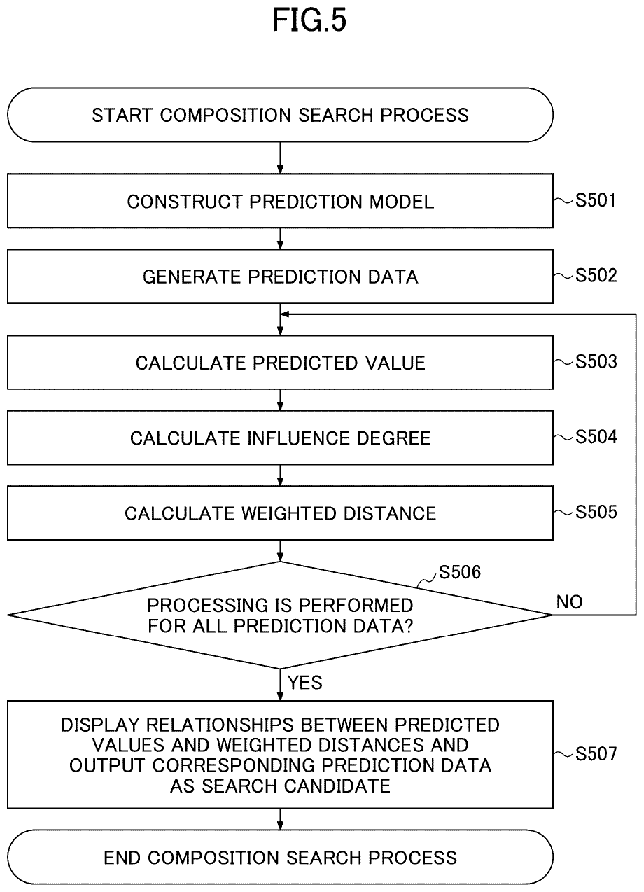

Next, a flow of a composition search process in the composition search system 100 will be described. FIG. 5 is a first flowchart illustrating the flow of the composition search process.

In step S501, the learning device 110 constructs the prediction model. As described above, in the present embodiment, the training data used when the learning device 110 constructs the prediction model includes a set of the parameters (the type, the preparation ratio, and the feature) of the raw material and the measured value of the physical property for multiple experimental samples (see FIG. 3 (A)).

Additionally, as described above, the prediction model constructed by the learning device 110 is a learned model obtained by performing machine learning using the training data in which the parameter of the raw material of the training data is the explanatory variable and the measured value of the physical property is the objective variable.

In step S502, the predicting device 120 generates the prediction data. As described above, the prediction data generated by the predicting device 120 in the present embodiment includes data of combinations of compositions exhaustively generated according to the constraint condition defining the upper and lower limits and the step size of the composition ratio, the raw materials that cannot be used at the same time, and the like or the features related to the compositions (see FIG. 3 (B)).

In step S503, the predicting device 120 calculates the predicted value from the prediction data by using the prediction model constructed in step S501.

In step S504, the predicting device 120 calculates the influence degree of each explanatory variable on the prediction by using the training data and the prediction model.

In step S505, the predicting device 120 calculates the weighted distance of the prediction data to the training data by using the influence degrees calculated in step S504.

In step S506, the predicting device 120 checks whether the predicted value and the weighted distance have been calculated for all the prediction data. If the predicted value and the weighted distance have been calculated for all the prediction data (YES in step S506), the process proceeds to step S507. If there is prediction data for which the predicted value and the weighted distance have not been calculated (NO in step S506), the process returns to step S503.

In step S507, the predicting device 120 displays multiple relationships between the predicted values and the weighted distances, and outputs the corresponding prediction data as the search candidate. As described above, when displaying multiple relationships between the predicted values and the weighted distances, the predicting device 120 plots and displays the predicted values on a two dimensional graph in which the horizontal axis represents the weighted distance and the vertical axis represents the predicted value (see FIG. 4).

### Effects of Composition Search Method According to First Embodiment

Next, the effects of the composition search method according to the first embodiment will be described. In the case of the composition search method according to the first embodiment, the user can select the search candidate in consideration of the predicted value and the weighted distance of the prediction data with respect to the training data.

To begin with, in the prediction of the value of the physical property, if a difference in an important parameter among information related to the composition is great, the actual values of the physical property are highly likely to greatly differ. With respect to the above, if the difference in the important parameter is great, the reliability of the predicted value predicted by the predicting device 120 is reduced.

Here, the unweighted distance is not suitable to be used as an index of the reliability of the predicted value because the important parameter is buried in the information related to the composition and is uniformly handled. That is, the weighted distance used in the first embodiment is more appropriate as an index indicating whether the reliability of the predicted value is higher or more challenging than the unweighted distance. As a result, according to the first embodiment, for example, by selecting a composition with a long weighted distance, the user can obtain a challenging search candidate for which focused searching in the important parameter is performed.

As described above, according to the first embodiment, because the search candidate can be selected while balancing the level of the reliability and the level of the challenge property of the predicted value, the composition for obtaining the target physical property value can be more efficiently searched for.

### Second Embodiment

Next, a composition search method according to a second embodiment will be described focusing on differences from the first embodiment.

### System Configuration of Composition Search System

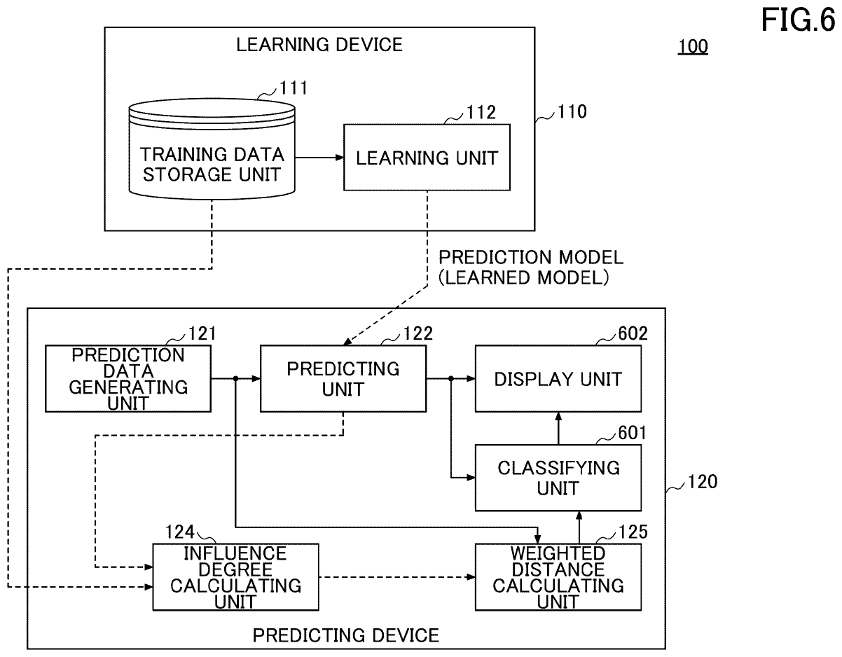

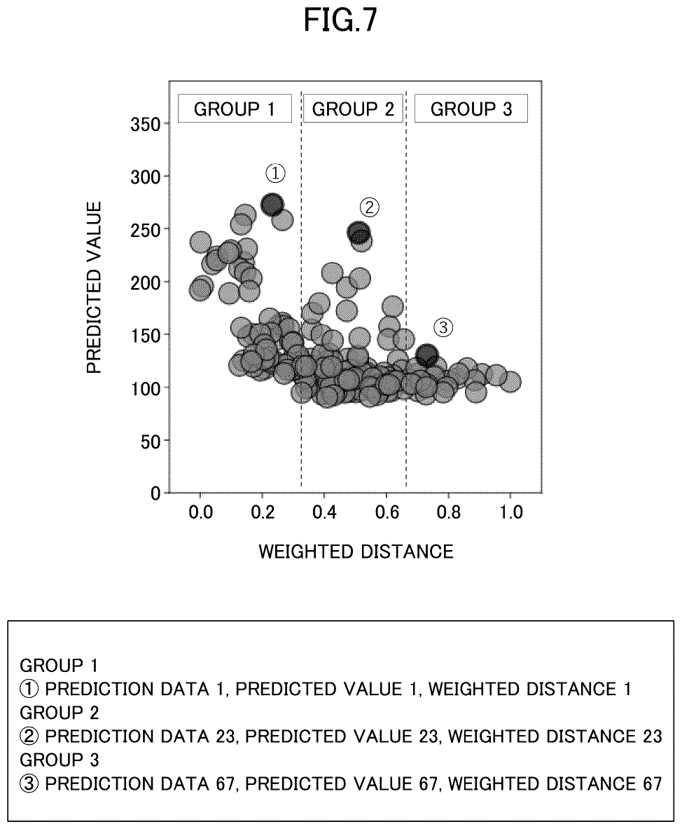

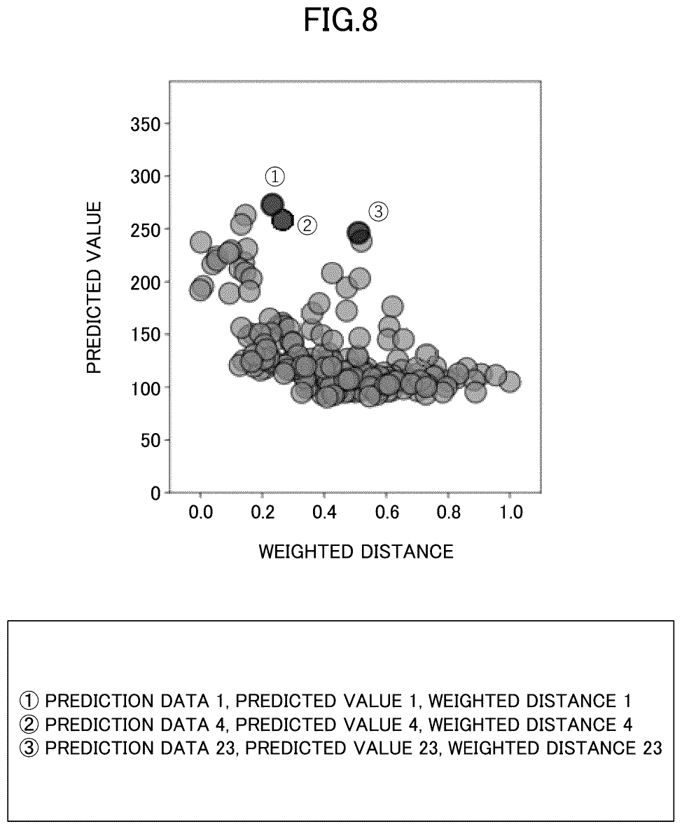

First, a system configuration of a composition search system that realizes the composition search method according to the second embodiment will be described using FIG. 6 with reference to FIG. 7 and FIG. 8. FIG. 6 is a second diagram illustrating an example of the system configuration of the composition search system. FIG. 7 and FIG. 8 are second and third diagrams illustrating examples of the graph indicating the relationship between the predicted value and the weighted distance.

The differences from the system configuration described with reference to FIG. 1 in the first embodiment are that, in the case of the system configuration illustrated in FIG. 6, the predicting device 120 includes a classifying unit 601, and a function of a display unit 602 is different from the function of the display unit 123.

The classifying unit 601 groups the predicted values calculated by the predicting unit 122 based on the weighted distance of the prediction data with respect to the training data. Additionally, the classifying unit 601 notifies the display unit 602 of a result of the grouping. Here, the grouping method by the classifying unit 601 may be selected suitably, and for example, either a method of equally dividing the weighted distance by a predetermined value between zero and one or a method of dividing the weighted distance such that the number of data in each group after the dividing is identical may be selected. Additionally, the number of groups may be a number set in advance or a number set by the user.

Additionally, the classifying unit 601 calculates an acquisition function serving as a reference when determining whether the prediction data is the search candidate, and notifies the display unit 602 of a result of the calculating. Specifically, the classifying unit 601 calculates the acquisition function using the following Equation (4), for example.

$$\begin{matrix}[ Eq . 4 ] & \\ Acq ( {X}_{i} ) = ( 1 - {s}_{g} ) * f ( {X}_{i} ) + {s}_{g} * {D}_{i} & ( 4 )\end{matrix}$$

$$( 0 \leq {s}_{g} \leq 1 )$$

Here, Xᵢ is the i-th prediction data, Acq (Xᵢ) is the acquisition function of the i-th prediction data, f (Xᵢ) is a value obtained by scaling the predicted value of the i-th prediction data to a value between zero and one, inclusive, S_{g} is a weighting factor in the g-th group, and Dᵢ is the weighted distance of the i-th prediction data to the training data. S_{g} may be set to 0 in all the groups. In that case, the acquisition function Acq (Xᵢ) is equal to the predicted value f (Xᵢ). s_{g} can be set by the user, and when s_{g} is not 0 in all the groups, the candidate selection can be achieved in which consideration is given to the weighted distance (Dᵢ) with respect to the training data in the group.

The display unit 602 displays multiple relations between the predicted values and the weighted distances, and outputs the corresponding prediction data as the search candidate in the order in which the acquisition function is higher for each group. Specifically, the prediction data (the information related to the composition) is selected from each group in the order in which the acquisition function is higher, and is output as the search candidate.

Here, the number of the search candidates output from each group can be appropriately set for each group, and can be set by the user in consideration of an experimental environment. For example, the user may set it such that the search candidates are equally output in each group. Alternatively, the user may set it such that the number of the search candidates output from a group having a long weighted distance is greater. In this case, the search can be performed with an emphasis on a composition having a long weighted distance with respect to the training data.

The example of FIG. 7 indicates a state in which, when multiple relationships between the predicted values and the weighted distances are displayed, the predicted values are plotted on a two dimensional graph with the weighted distances on the horizontal axis and the predicted values on the vertical axis, and the predicted values for which the acquisition function is high are displayed with numbering, and the corresponding prediction data is output as the search candidate.

Here, the above description assumes that the classifying unit 601 groups the prediction values and calculates the acquisition function, and the display unit 602 displays the predicted value for which the acquisition function is high with numbering for each group and outputs the corresponding prediction data as the search candidate.

However, the functions of the classifying unit 601 and the display unit 602 are not limited to this, and for example, the classifying unit 601 may be configured to calculate the acquisition function without grouping the predicted values, and the display unit 602 may be configured to display, with numbering, the predicted values for which the acquisition function is high and output the corresponding prediction data as the search candidate.

In this case, the classifying unit 601 may select the prediction data based on an acquisition function calculated using, for example, the following Equation (5) or Equation (6), and output the prediction data as the search candidate.

$$\begin{matrix}[ Eq . 5 ] & \\ Acq ( {X}_{i} ) = ( 1 - α ) * f ( {X}_{i} ) + α * {D}_{i} & ( 5 )\end{matrix}$$

$$( 0 \leq α \leq 1 )$$

$$\begin{matrix}[ Eq . 6 ] & \\ Acq ( {X}_{i} ) = ( 1 - α ) * f ( {X}_{i} ) + α * ( 1 - {D}_{i} ) & ( 6 )\end{matrix}$$

$$( 0 \leq α \leq 1 )$$

Here, Xᵢ is the i-th prediction data, Acq (Xᵢ) is the acquisition function of the i-th prediction data, f (Xᵢ) is a value obtained by scaling the predicted value of the i-th prediction data to a value between zero and one, inclusive, Dᵢ is the weighted distance of the i-th prediction data with respect to the training data, and a is the weighting factor in Dᵢ.

According to the classifying unit 601, the user can adjust which of the predicted value f (Xᵢ) and the weighted distance Dᵢ or 1-Dᵢ is to be emphasized by appropriately setting the weighting factor α included in the acquisition function. For example, in the case of Equation (5), when α is increased, a high predicted value f (Xᵢ) can be searched for, while putting an emphasis on a composition having a long weighted distance with respect to the training data. Conversely, in the case of Equation (6), when α is decreased, a high predicted value f (Xᵢ) can be searched for, while putting an emphasis on a composition having a close weighted distance with respect to the training data and a high reliability of the predicted value.

Additionally, in the case of the classifying unit 601 described above, the display unit 602 selects the prediction data in the order in which the acquired function is higher and outputs the prediction data as the search candidate (see FIG. 8). Here, the display unit 602 can use either Equation (5) or Equation (6), or both as the acquisition function. When both of the equations are used, the number of the search candidates to be output by each of the equations may be appropriately set in consideration of the total number of the search candidates to be output.

### Flow of Composition Search Process in Composition Search System

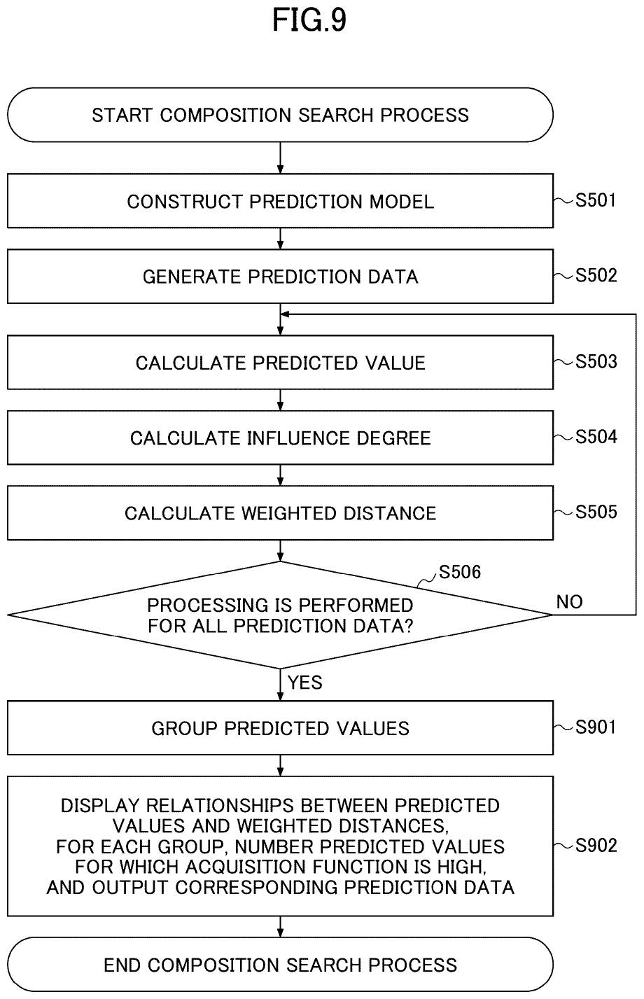

Next, the flow of the composition search process in the composition search system 100 will be described. FIG. 9 is a second flowchart illustrating the flow of the composition search process.

Here, in FIG. 9, the processing from step S501 to step S506 is substantially the same as the processing described in the first embodiment with reference to FIG. 5, and the description thereof will be omitted here.

In subsequent step S901, the predicting device 120 groups the prediction values by the weighted distances.

In step S902, the predicting device 120 displays the relationships between the predicted values and the weighted distances, and outputs the corresponding prediction data as the search candidate in the order in which the acquisition function is higher for each group. When displaying the relationships between the prediction values and the weighted distances, as illustrated in FIG. 7, the predicting device 120 plots the prediction values on a two dimensional graph with the weighted distance on the horizontal axis and the prediction value on the vertical axis, and then numbers and displays the prediction values for which the acquisition function is high, and outputs the corresponding prediction data as the search candidates.

### Conclusion

As is clear from the above description, in the composition search method according to the second embodiment, the predicted values are grouped by the weighted distances, and the relationships between the predicted values and the weighted distances are displayed. With this, according to the composition search method of the second embodiment, the prediction data for a high predicted value can be selected in the level of the challenge property for each group and the prediction data can be output as the search candidates.

Additionally, in the composition search method according to the second embodiment, the acquisition function of the prediction data is calculated, and the prediction data corresponding to the predicted value for which the calculated acquisition function is high is output as the search candidate. With this, according to the composition search method of the second embodiment, the search candidate can be output while balancing the level of the reliability and the level of the challenge property of the predicted value.

### Third Embodiment

Subsequently, a composition search method according to a third embodiment will be described focusing on differences from the first and second embodiments described above.

### System Configuration of Composition Search System

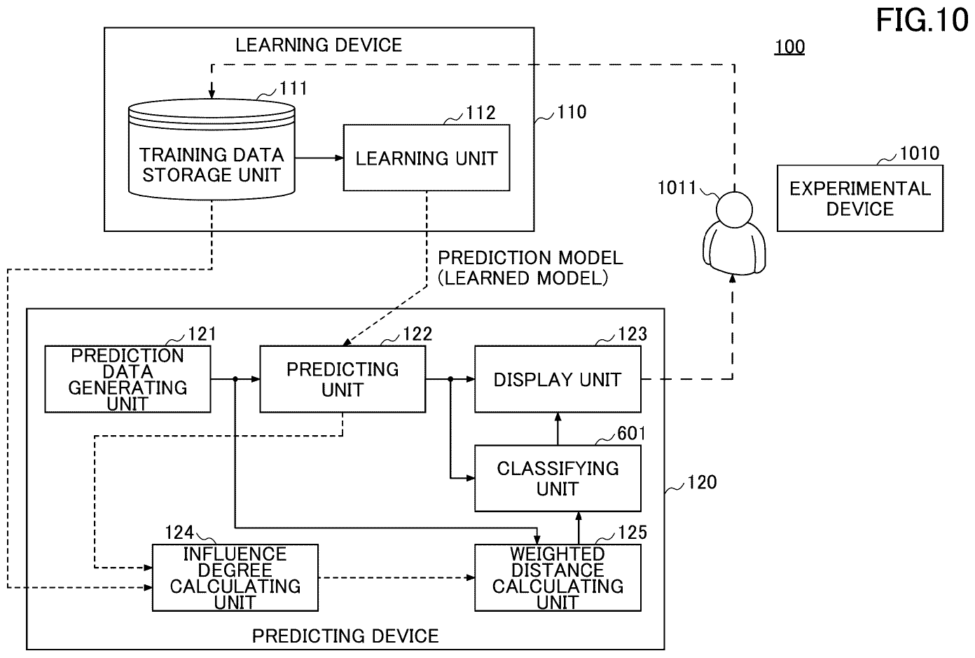

First, a system configuration of a composition search system for realizing the composition search method according to the third embodiment will be described using FIG. 10. FIG. 8 is a third diagram illustrating an example of the system configuration of the composition search system.

A difference from the system configuration described using FIG. 6 in the second embodiment is that the system configuration illustrated in FIG. 10 includes an experimental device 1010.

The experimental device 1010 is a device used when an experimenter 1011 evaluates the physical property with respect to a composition of the output search candidate. The experimenter 1011 confirms whether the value of the physical property obtained by evaluating the physical property by using the experimental device 1010 reaches the target value, and ends the search for the composition if the target value is reached. If the target value is not reached, the experimenter 1011 adds, to the training data, a set of information related to the composition of the search candidate on which the experiment has been performed and the obtained value of the physical property, and stores the training data in the training data storage unit 111.

### Flow of Composition Search Process in Composition Search System

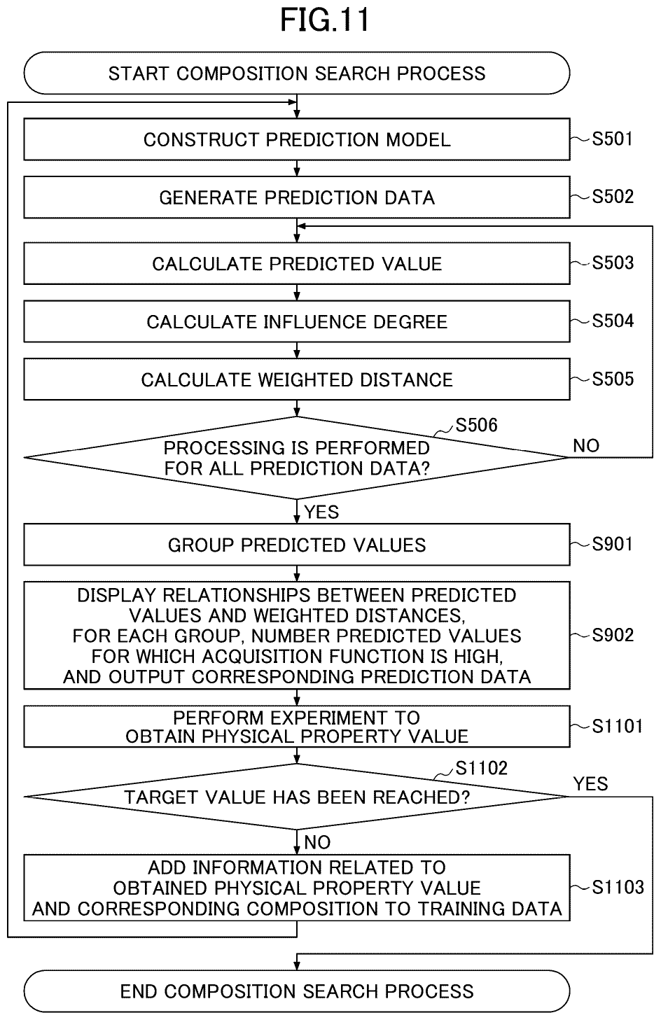

Next, the flow of the composition search process in the composition search system 100 will be described. FIG. 11 is a third flowchart illustrating the flow of the composition search process.

Here, in FIG. 11, the processing from step S501 to step S902 is substantially the same as the processing described using FIG. 9 in the second embodiment, and the description thereof will be omitted here.

In subsequent step S1101, the experimenter 1011 uses the experimental device 1010 to evaluate the physical property with respect to the composition of the search candidate output in step S902 by using the experimental device 1010, and obtains the value of the physical property.

In step S1102, the experimenter 1011 confirms whether the value of the physical property obtained in step S1101 reaches the target value. If the target value is reached (YES in step S1102), the search for the composition is ended. If the target value is not reached (NO in step S1102), the process proceeds to step S1103.

In step S1103, the experimenter 1011 adds, to the training data, the set of the information related to the composition of the search candidate on which the experiment has been performed in step S1101 and the obtained value of the physical property, and then returns to step S501. In the composition search system 100, respective steps of step S501 to step S1103 described above are repeated until the value of the physical property reaches the target value in the step S1102 by using the updated training data.

### Conclusion

As is clear from the above description, in the composition search method according to the third embodiment, the physical property is evaluated with respect to the composition of the search candidate, and when the value of the physical property does not reach the target value, the set of the information related to the composition of the search candidate and the obtained value of the physical property is added to the training data.

As described, by using the configuration to evaluate the physical property by the experiment with respect to the search candidate in which the level of the reliability and the level of the challenge property of the predicted value are balanced, the number of experiments until the value of the physical property reaches the target value can be reduced.

Here, in the above description, the process when the value of the physical property reaches the target value is not mentioned, but when the value of the physical property reaches the target value, for example, the material is designed and produced based on the corresponding search candidate. This enables a material having the target physical property to be designed and produced.

### EXAMPLE

In the following, a specific example of the composition search method according to the third embodiment among the above-described embodiments will be described.

In the present example, a dataset of the paper of Turab Lookman et al. (https://www.nature.com/articles/s41598-018-21936-3 #Sec12)), in which a composition of a metallic compound, a feature related to the composition, and a physical property are described, is used as the training data and the prediction data. The dataset is a modulus dataset for 223 M₂AX chemical compound compositions (M: a transition metal, A: a p-block element, X: nitrogen (N) or carbon (C)), some of which are indicated in Table 1. p, d, and s orbital radii of each element in the element sites (M, A, and X) are described in the second column to the eighth column of Table 1, and these are used as the explanatory variables of the training data and the prediction data. Additionally, the Young's modulus in the ninth column is used as the objective variable of the training data.

TABLE 1

| | M-atom | M-atom | M-atom | A-atom | A-atom | X-atom | X-atom 0 | |
|---|---|---|---|---|---|---|---|---|
| | p- | d- | s- | s- | p- | s- | p- | |
| | orbital | orbital | orbital | orbital | orbital | orbital | orbital | Young's |
| | radii | radii | radii | radii | radii | radii | radii | modulus |
| 1 | 0.5 | 0.539 | 1.57 | 0.445 | 1.184 | 0.62 | 0.596 | 92 |
| 2 | 0.5 | 0.539 | 1.57 | 1.06 | 1.319 | 0.62 | 0.596 | 135 |
| 3 | 0.5 | 0.539 | 1.57 | 1.093 | 1.382 | 0.62 | 0.596 | 135 |
| 4 | 0.5 | 0.539 | 1.57 | 1.01 | 1.215 | 0.62 | 0.596 | 142 |
| 5 | 0.5 | 0.539 | 1.57 | 1.044 | 1.312 | 0.62 | 0.596 | 140 |
| 6 | 0.5 | 0.539 | 1.57 | 0.96 | 1.254 | 0.62 | 0.596 | 133 |
| 7 | 0.5 | 0.539 | 1.57 | 0.445 | 1.184 | 0.521 | 0.4875 | 106 |
| 8 | 0.5 | 0.539 | 1.57 | 1.027 | 1.24 | 0.62 | 0.596 | 154 |
| 9 | 0.5 | 0.539 | 1.57 | 1.01 | 1.215 | 0.521 | 0.4875 | 165 |
| | . . . | . . . | . . . | . . . | . . . | . . . | . . . | . . . |
| 223 | 0.599 | 0.784 | 1.413 | 0.803 | 0.9175 | 0.521 | 0.4875 | 315 |

The search for the optimum composition by repeating the output (the selection and proposal) of the search candidate and the evaluation (the measurement) of the physical property by the experiment was reproduced by Example 1 and Comparative Examples 1 and 2 by using the dataset described above. Specifically, the numbers of times until a composition in which the Young's modulus is the highest is found in the dataset are compared. It can be said that as the number of times becomes smaller, the method can search for the optimum composition more efficiently.

Example 1 indicates a case of performing the composition search according to the flowchart of FIG. 11, which is the composition search method according to the third embodiment. In order to compare the effects of weighting, Comparative Example 1 indicates a case of performing the composition search without performing the processing of steps S504 and S505 in the flowchart of FIG. 11.

Additionally, Comparative Example 2 indicates a case of performing the composition search by a composition search method of simply outputting corresponding prediction data as the search candidates in the order in which the predicted value is high, without considering the distance from the training data.

In the following, the procedure of Example 1 will be described specifically.

In steps S501 and S502, the learning device 110 extracts, as the training data to be used first, a combination of the orbital radii and the Young's modulus of each of 24 elements having low Young's modulus among the 223 chemical compound compositions included in the dataset. Additionally, the learning device 110 sets the remaining 199 chemical compound compositions included in the dataset as the explanatory variables (the orbital radius of the respective elements) of the prediction data. The learning device 110 then performs learning using a random forest regression model of scikit-learn as a technique of the prediction model to construct the prediction model.

In step S503, the predicting device 120 calculates the predicted value from the prediction data by using the prediction model constructed in step S501.

In step S504, the predicting device 120 calculates Gini importance included in scikit-learn as the influence degree.

In step S505, the predicting device 120 calculates the weighted distances by using the influence degree calculated in step S504. The predicting device 120 repeats the steps S503 to S506 to calculate the predicted values and the weighted distances for all the prediction data, and then proceeds to step S901.

In step S901, the predicting device 120 groups the prediction data according to the weighted distances. Here, the weighted distances are divided into three groups by a method of dividing the weighted distances by a predetermined numerical value.

In step S902, the predicting device 120 outputs one composition from each group as the search candidate. Specifically, the predicting device 120 uses the above-described Equation (4) as the acquisition function, sets s_{g} to 0 in all groups, and outputs the corresponding prediction data as the search candidate in the order in which the acquisition function is high in each group.

In step S1101, the experimenter 1011 acquires Young's modulus corresponding to the output search candidate (=the prediction data) from the dataset, instead of performing the experiment and measurement on the output search candidate.

In step S1102, the experimenter 1011 confirms whether the Young's modulus acquired in step S1101 reaches the target value (the highest value in the dataset). If the Young's modulus reaches the target value, the search is ended, and the number of times for ending of the search is obtained. If the Young's modulus does not reach the target value, the process proceeds to the next step S1103.

In step S1103, the experimenter 1011 adds a set of the information related to the output composition of the search candidate and the obtained value of the physical property to the training data for updating, and returns to step S501 of constructing the prediction model. The experimenter 1011 has repeated the above steps until the Young modulus reaches the target value in step S1102. That is, by adopting one search candidate from each group, the prediction data is reduced by three as a whole of the three groups, and the orbital radius and the corresponding Young's modulus of each element, which are the prediction data, are added to the training data.

Here, the random forest regression model used in Example 1 has randomness in search, and it is conceivable that a search candidate having the highest Young's modulus may be found for the first time by chance. Therefore, in order to appropriately compare the numbers of times until the search ends, in Example 1, Comparative Example 1, and Comparative Example 2, the procedure until the target value is reached in the step S1102 described above is repeated 100 times to acquire 100 numbers of times for ending of the search, and average values and standard deviations thereof are calculated and compared.

A difference between the procedure of Comparative Example 1 and that of Example 1 will be described specifically.

In Comparative Example 1, the processing corresponding to step S503 in Example 1 is not performed, and distances that are not weighted are calculated by setting all the influence degrees wₜ of the explanatory variables in the above Equation (2) to 1 in step S504. Additionally, in step S901, the distances that are not weighted are used instead of the weighted distances. The other procedures are performed as in Example 1.

A difference between the procedure of Comparative Example 2 and that of Example 1 will be described specifically.

In Comparative Example 2, the processing corresponding to steps S503, S504, S901, and S1101 in Example 1 is not performed, and three corresponding prediction data are output as the search candidates in the order in which the predicted value obtained in step S502 is higher, and then step S1101 is performed. The other procedures performed as in Example 1.

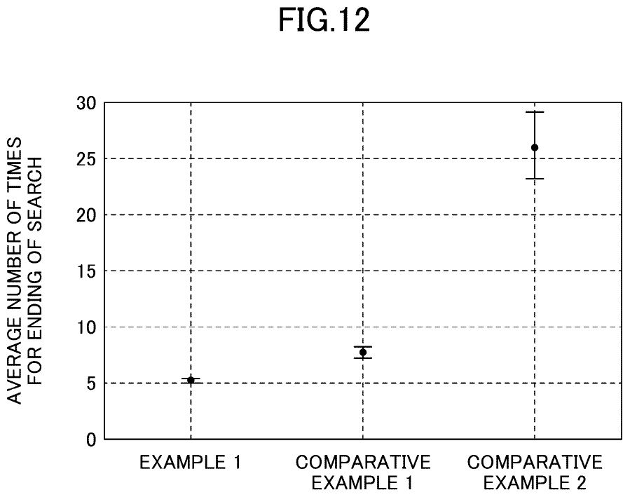

Results are indicated in Table 2 and FIG. 12. The average number of times for ending of the search was 5.2 times in Example 1, 7.7 times in Comparative Example 1, and 26.0 times in Comparative Example 2, and Example 1 indicates the smallest number of times. Table 2 indicates the average value and standard deviation of the numbers of times for ending of the search. The average numbers of times for ending of the search in Example 1 and Comparative Examples 1 and 2 are plotted in FIG. 12, and the standard deviations are indicated as error bars.

Because a value of the average number of times for ending of the search in Comparative Example 2 is clearly large, it can be said that Comparative Example 2 is less efficient than Example 1 and Comparative Example 1. A difference between the results of Example 1 and Comparative Example 1 was tested by the null hypothesis, which is a hypothesis that the effect does not exist if there is no difference between two groups. The null hypothesis is that there is no difference in the average value between the two groups. As a specific statistical method, Student's t-test was performed. As a result of the test, the p value was less than or equal to 0.01, which is the significance level, and the null hypothesis was rejected. It was determined that there was a significant difference in the number of times for ending of the search between Example 1 and Comparative Example 1 at the significance level 18. This confirms that the composition search method according to the third embodiment is a method that can efficiently search for the composition.

TABLE 2

| | AVERAGE NUMBER OF TIMES |
|---|---|
| | FOR ENDING OF SEARCH |
| | (STANDARD DEVIATION) |
| EXAMPLE 1 | 5.2(±1.4) |
| COMPARATIVE EXAMPLE 1 | 7.7(±2.4) |
| COMPARATIVE EXAMPLE 2 | 26.0(±14.5) |

This application claims priority to Japanese Patent Application No. 2021-163338 filed on Oct. 4,2021, the entire contents of which are incorporated herein by reference.

### INDUSTRIAL AVAILABILITY

The composition search method of the present invention can be used for the material design in alloy materials, organic materials, composite materials, and the like.

1. A composition search method for a material, comprising:
constructing a prediction model by learning training data in which information related to a composition of a material is set as an explanatory variable and a value of a physical property of the material is set as an objective variable;
calculating a predicted value of the physical property by inputting, into the prediction model, prediction data for newly searching for a composition;
calculating an influence degree of each explanatory variable on prediction by using the training data and the prediction model;
calculating a weighted distance of the prediction data with respect to the training data by using the influence degree; and
displaying a relationship between the predicted value and the weighted distance, and outputting corresponding prediction data as a search candidate.

2. The composition search method as claimed in claim 1, wherein the calculating of the weighted distance includes scaling the weighted distance to a value between zero and one, inclusive.

3. The composition search method as claimed in claim 1,
wherein the prediction data are a combination of information related to the composition exhaustively generated according to a constraint condition of a step size or a composition ratio that is set in advance, and
wherein the displaying of the relationship between the predicted value and the weighted distance includes displaying a plurality of said relationships between the said calculated predicted values and the said weighted distances, by repeating the calculating of the predicted value of the physical property to the calculating of the weighted distance.

4. The composition search method as claimed in claim 3, further comprising grouping the predicted values by the weighted distances, and
wherein the displaying of the relationship between the predicted value and the weighted distance includes dividing the prediction data into groups and output the divided prediction data.

5. The composition search method as claimed in claim 4, wherein the displaying of the relationship between the predicted value and the weighted distance includes outputting corresponding prediction data as search candidates in an order in which the predicted value is higher for each of the groups.

6. The composition search method as claimed in claim 4, wherein, the grouping is performed by equally dividing the weighted distances by a predetermined value between zero and one.

7. The composition search method as claimed in claim 4, wherein, the grouping is performed by dividing the weighted distance between zero and one, such that a number of the predicted values in a group after the division is identical.

8. The composition search method as claimed in claim 3, wherein in the displaying of the relationship between the predicted value and the weighted distance, a number of the prediction data to be output as the search candidate is set by a user.

9. The composition search method as claimed in claim 4, further comprising:
calculating an acquisition function Acq(Xᵢ) with respect to the predicted value and the weighted distance calculated from the prediction data by using the following Equation (1); and
outputting corresponding prediction data as the search candidates in an order in which the calculated acquisition function is higher,
$$Acq ( {X}_{i} ) = ( 1 - {s}_{g} ) * f ( {X}_{i} ) + {s}_{g} * {D}_{i} \qquad ( 1 )$$
$$( 0 \leq {s}_{g} \leq 1 )$$
where Xᵢ is the i-th prediction data, f(Xᵢ) is a predicted value of Xᵢ scaled to a value between zero and one, inclusive, s_{g} is a weighting factor in the g-th group, and Dᵢ is the weighted distance of Xᵢ.

10. The composition search method as claimed in claim 3, further comprising:
performing an experiment based on the information related to the composition of the prediction data output as the search candidate in the outputting, to obtain a value of the physical property; and
adding the information related to the composition corresponding to the obtained value of the physical property to the training data,
wherein processing of constructing the prediction model by using the training data to which data is added in the constructing the prediction model to processing of obtaining the value of the physical property in the obtaining the value of the physical property are repeated until the obtained value of the physical property reaches a predetermined target value.
